(19) United States
(12) Patent Application Publication
(10) Pub. No.: US 2005/0255178 A1
(43) Pub. Date: Nov. 17, 2005

(54) Enhancing the effectiveness of an inhaled therapeutic gas

(76) Inventors: Kenneth D. Bloch, Brookline, MA (US); Fumito Ichinose, Brookline, MA (US); Warren M. Zapol, Cambridge, MA (US); Oleg V. Evgenov, Beverly, MA (US)

Correspondence Address: FISH & RICHARDSON PC, P.O. BOX 1022, MINNEAPOLIS, MN, 55440-1022, US

(21) Appl. No.: 11/050,959

(22) Filed: Feb. 4, 2005

Related U.S. Application Data
(60) Provisional application No. 60/542,000, filed on Feb. 4, 2004.

(51) Int. Cl. A61K 33/00

(52) U.S. Cl. 424/718

(57) ABSTRACT

Methods for enhancing the therapeutic or prophylactic effectiveness of an inhaled therapeutic gas are disclosed. The methods include administering to a mammal by inhalation a therapeutically effective amount of gaseous nitric oxide or carbon monoxide, and administering to the mammal a composition containing a compound that sensitizes soluble guanylate cyclase.

### CROSS REFERENCE TO RELATED APPLICATIONS

This application claims priority from Provisional Application No. 60/542,000, filed Feb. 4, 2004. The prior application is incorporated herein by reference in its entirety.

### STATEMENT AS TO FEDERALLY SPONSORED RESEARCH

This invention was made with Government support under grant numbers HL57172 and HL42397 awarded by the National Institutes of Health/National Heart, Lung, and Blood Institute. The Government may have certain rights in the invention.

### FIELD OF THE INVENTION

This invention relates to compositions and methods for enhancing the effectiveness of therapeutic gases.

### BACKGROUND

Nitric oxide (NO) is a cell membrane-permeable, free radical molecule which accounts for the vasodilator activity of endothelium-derived relaxing factor (reviewed in Schmidt et al., Cell 78:919-925 [1994]). NO interacts with several intracellular molecular targets, one of which is soluble guanylate cyclase (sGC). Binding of NO to the heme group in sGC stimulates the conversion of guanosine triphosphate (GTP) to guanosine-3′,5′-cyclic monophosphate (cGMP). cGMP exerts it effects on cells, in part, through its action on cGMP-dependent protein kinase (cGDPK). Additional cGMP targets include cGMP-gated ion channels and cGMP-regulated cyclic nucleotide phosphodiesterases. Phosphodiesterases (PDEs) inactivate cGMP by converting it to GMP.

The biological effects of NO are also mediated by cGMP-independent mechanisms. NO can serve as an antioxidant, opposing the effect of superoxides. The antioxidant properties of NO appear to account for its ability to modulate proinflammatory activation of endothelial cells. NO may also react with superoxide to form peroxynitrite which may be responsible for the cellular toxicity associated with high levels of NO production.

### SUMMARY

The invention is based, at least in part, on the discovery that a compound that sensitizes soluble guanylate cyclase can augment and/or prolong the therapeutic effectiveness of an inhaled therapeutic gas. The therapeutic gases described herein are nitric oxide and carbon monoxide.

In one aspect, the invention features a method for enhancing the therapeutic or prophylactic effectiveness of inhaled NO, the method including: (1) identifying a mammal (e.g., a human) that has or is at risk of developing a condition amenable to treatment or prevention by inhalation of gaseous NO; (2) administering to the mammal by inhalation a therapeutically effective amount of gaseous NO; and (3) administering to the mammal a composition containing a compound that sensitizes soluble guanylate cyclase, wherein the composition contains an amount of the compound sufficient to enhance the therapeutic or prophylactic effectiveness of the inhaled gaseous NO. In some embodiments, the method does not include the administration to the mammal of superoxide dismutase.

The invention also features a method for enhancing the therapeutic or prophylactic effectiveness of inhaled carbon monoxide (CO), the method including: (1) identifying a mammal (e.g., a human) that has or is at risk of developing a condition amenable to treatment or prevention by inhalation of gaseous CO; (2) administering to the mammal by inhalation a therapeutically effective amount of gaseous CO; and (3) administering to the mammal a composition containing a compound that sensitizes soluble guanylate cyclase, wherein the composition contains an amount of the compound sufficient to enhance the therapeutic or prophylactic effectiveness of the inhaled gaseous CO. In some embodiments, the method does not include the administration to the mammal of superoxide dismutase.

As detailed herein, inhalation of CO either alone or in combination with BAY 41-2272 (a compound that sensitizes soluble guanylate cyclase) was found to have no vasodilator effect on experimentally induced pulmonary vasoconstriction. However, for those indications (e.g., treating or preventing ischemia-reperfusion injury or inflammation, extending the survival of an organ transplant, and inhibiting chronic rejection in a recipient) for which CO inhalation alone has been determined to have a beneficial effect, co-administration of a compound that sensitizes soluble guanylate cyclase is expected to enhance the therapeutic effectiveness of inhaled CO for those indications.

In some embodiments, prior to administering the therapeutic gas and the composition, the mammal is diagnosed as having pulmonary vasoconstriction. In other embodiments, prior to administering the therapeutic gas and the composition, the mammal is diagnosed as being at risk of developing pulmonary vasoconstriction. The pulmonary vasoconstriction can be, for example, acute pulmonary vasoconstriction, reversible pulmonary vasoconstriction, chronic pulmonary vasoconstriction which has a reversible component, or chronic pulmonary vasoconstriction which does not have a reversible component.

The mammal can have or be at risk of developing pneumonia, traumatic injury, aspiration or inhalation injury, fat embolism in the lung, acidosis, inflammation of the lung, adult respiratory distress syndrome, acute mountain sickness, post cardiac surgery acute pulmonary hypertension, persistent pulmonary hypertension of the newborn, perinatal aspiration syndrome, hyaline membrane disease, acute pulmonary thromboembolism, acute pulmonary edema, heparin-protamine reactions, sepsis, hypoxia, asthma, status asthmaticus, or hypoxia of the newborn.

The mammal can have or be at risk of developing chronic pulmonary hypertension, bronchopulmonary dysplasia, chronic pulmonary thromboembolism, idiopathic pulmonary hypertension, or chronic hypoxia.

In some embodiments, prior to administering the therapeutic gas and the composition, the mammal is diagnosed as having bronchoconstriction.

In other embodiments, prior to administering the therapeutic gas and the composition, the mammal is diagnosed as being at risk of developing bronchoconstriction. The bronchoconstriction can be, for example, associated with asthma.

In some embodiments, prior to administering the therapeutic gas and the composition, the mammal is diagnosed as having a vascular thrombosis. In other embodiments, prior to administering the therapeutic gas and the composition, the mammal is diagnosed as being at risk of developing a vascular thrombosis. The vascular thrombosis can be, for example, an arterial thrombosis or a venous thrombosis. In some cases, the vascular thrombosis is not a pulmonary thrombosis.

In some embodiments, prior to administering the therapeutic gas and the composition, the mammal is diagnosed as having or being at risk of developing an acute ischemic coronary syndrome. The acute ischemic coronary syndrome can be, for example, myocardial infarction, unstable angina pectoris, thrombosis after coronary revascularization, or reocclusion after coronary thrombolysis. The acute ischemic coronary syndrome can be associated with an artery-occluding disease. The acute ischemic coronary syndrome can be associated with a vascular interventional procedure (e.g., angioplasty such as percutaneous transluminal coronary angioplasty (PTCA), coronary artery bypass surgery, or a procedure to implant a coronary artery stent).

In some embodiments, prior to administering the therapeutic gas and the composition, the mammal is diagnosed as having arterial restenosis. In other embodiments, prior to administering the therapeutic gas and the composition, the mammal is diagnosed as being at risk of developing arterial restenosis. In some cases, the mammal has undergone or is preparing to undergo a vascular interventional procedure (e.g., angioplasty such as PTCA, coronary artery surgery, or a procedure to implant a coronary artery stent).

In those methods that involve a vascular interventional procedure to implant a stent, such a stent can optionally be coated with a compound such as an antiproliferative agent or a an agent that sensitizes soluble guanylate cyclase to NO (thereby sensitizing NO-exposed platelets and leukocytes which adhere to them).

In some embodiments, prior to administering the therapeutic gas and the composition, the mammal is diagnosed as having a hemoglobinopathy. In other embodiments, prior to administering the therapeutic gas and the composition, the mammal is diagnosed as being at risk of developing a hemoglobinopathy.

The hemoglobinopathy can be characterized by (a) a reduced affinity of the patient's hemoglobin for oxygen compared with the affinity for oxygen of normal adult hemoglobin (Hb-A), or (b) a tendency of the patient's erythrocytes to sickle. In some cases, the hemoglobinopathy is selected from the group consisting of sickle cell trait; Hb-C, Hb-D, Hb-E, Hb-H, Hb-I, and Hb-Kansas disorders; or a combination of Hb-S with a second mutant β-globin allele. The hemoglobinopathy can be sickle cell disease.

In some embodiments, prior to administering the therapeutic gas and the composition, the mammal is diagnosed as having an ischemia-reperfusion injury. In other embodiments, prior to administering the therapeutic gas and the composition, the mammal is diagnosed as being at risk of developing ischemia-reperfusion injury. The ischemia-reperfusion injury can be in a non-pulmonary tissue. In some cases, the ischemia-reperfusion injury is caused by surgery, e.g., heart bypass surgery or transplantation surgery such as kidney transplantation surgery or heart transplantation surgery. In some cases, the ischemia-reperfusion injury occurs in the kidney, brain, or intestine. In some cases, the ischemia-reperfusion injury is caused by a stroke. In some cases, the ischemia-reperfusion injury occurs as a result of vascular occlusion at the time of aortic surgery. In other cases, the ischemia-reperfusion injury occurs as a result of re-vascularization of a limb.

In some embodiments, prior to administering the therapeutic gas and the composition, the mammal is diagnosed as having inflammation. In other embodiments, prior to administering the therapeutic gas and the composition, the mammal is diagnosed as being at risk of developing inflammation. The inflammation can be in a non-pulmonary tissue. The inflammation can be associated with arthritis, myocarditis, encephalitis, transplant rejection, systemic lupus erythematosis, gout, dermatitis, inflammatory bowel disease, hepatitis, or thyroiditis.

The invention also features a method of improving gas exchange in the lungs of a mammal, the method including: (1) identifying a mammal (e.g., a human) for whom an improvement in gas exchange within the lungs would be beneficial; (2) administering to the mammal by inhalation a therapeutically effective amount of gaseous NO; and (3) administering to the mammal a composition containing a compound that sensitizes soluble guanylate cyclase, wherein the composition contains an amount of the compound sufficient to enhance the therapeutic effectiveness of the inhaled gaseous NO and improve gas exchange in the lungs of a mammal. In some embodiments, the method does not include the administration to the mammal of superoxide dismutase.

In some embodiments, the mammal is hypoxic and/or suffers from a lung injury.

The composition containing a compound that sensitizes soluble guanylate cyclase can be introduced into the mammal by, for example, an oral, intravenous, intramuscular, subcutaneous, or intraperitoneal route. In addition, the composition can be introduced into the mammal by providing an aerosol or dry powder containing the composition for inhalation by the mammal. The composition can be inhaled in the therapeutic gas containing the gaseous NO or CO. Exemplary compounds that sensitize soluble guanylate cyclase are YC-1 (3-(5′-hydroxymethyl-2′-furyl)-1-benzylindazole) and BAY 41-2272 (3-(4-amino-5-cyclopropylpyrimidine-2-yl)-1-(2-fluorobenzyl)-1H-pyrazolo[3,4-b]pyridine).

NO gas inhaled by the mammal can be administered at a predetermined concentration. Preferably it is administered in the absence of tobacco smoke. The predetermined concentration can be, for example, 0.1 ppm to 300 ppm, 1 ppm to 250 ppm, or 5 ppm to 200 ppm. Alternatively, the predetermined concentration can be, for example, at least 5 ppm, at least 40 ppm, at least 80 ppm, or 180 ppm or less.

In some embodiments, the therapeutic gas is inhaled continuously for an extended period or inhaled intermittently for an extended period. For example, the therapeutic gas can be inhaled continuously for at least three minutes, after which inhalation can be stopped for a period of at least 0.5, 1, 2, 6, 12, or 24 hours prior to a subsequent inhalation.

An advantage of some of the methods described herein is the achievement of a desired therapeutic outcome by using an NO or CO dosage lower than that required if NO or CO alone were administered to a patient. Lower dosages of NO or CO are expected to decrease the likelihood of adverse events that may accompany inhalation of higher doses of these therapeutic gases.

Another advantage of some of the methods described herein is that the co-administration of a compound that sensitizes soluble guanylate cyclase unexpectedly results in the prolongation of a response to an inhaled therapeutic gas. Accordingly, certain methods described herein can facilitate long-term NO or CO therapy by permitting an intermittent inhalation of gaseous NO or CO by a patient.

Unless otherwise defined, all technical and scientific terms used herein have the same meaning as commonly understood by one of ordinary skill in the art to which this invention belongs. Suitable methods and materials are described below, although methods and materials similar or equivalent to those described herein can also be used in the practice or testing of the present invention. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIGS. 1A-1B are graphs depicting the effects of BAY 41-2272 on mean pulmonary arterial pressure (1A) and pulmonary vascular resistance (1B).

FIGS. 2A-2B are graphs depicting the effects of L-NAME on pulmonary vasodilation (2A) and systemic vasodilation (2B) induced by BAY 41-2272.

FIGS. 3A-3D are graphs depicting percent changes of pulmonary arterial pressure (3A), percent changes of pulmonary vascular resistance (3B), half-time of reversal of pulmonary vasodilation (T_{1/2}) (3C), and transpulmonary cGMP release during inhalation of NO alone (NO) or in combination with BAY 41-2272 (BAY+NO) (3D).

FIG. 4 is a graph depicting the effects of YC-1, inhaled NO, and the combination of YC-1 and inhaled NO on pulmonary and systemic arterial pressure.

FIG. 5 is a graph depicting the effects of inhaled NO and YC-1 administration on pulmonary vasodilation.

### DETAILED DESCRIPTION

Administration of Inhaled Nitric Oxide and Carbon Monoxide

Methods for safe and effective administration of NO and CO by inhalation are well known. See, e.g., Zapol, U.S. Pat. No. 5,570,683; Zapol et al., U.S. Pat. No. 5,904,938; Bach et al., U.S. Published Application No. 20030039638; and Frostell et al., 1991, Circulation 83:2038-2047. NO for inhalation is available commercially (INOmax™, INO Therapeutics, Inc., Clinton, N.J.).

A suitable starting dosage for NO administered by inhalation may be 20 ppm. See, e.g., INOmax™ package insert. However, dosage can vary, e.g., from 0.1 ppm to 100 ppm, depending on the age and condition of the patient, the disease or disorder being treated, amount of sensitizer of soluble guanylate cyclase administered to the patient, and other factors that the treating physician may deem relevant. Preferably, the lowest effective dose is inhaled. To arrive at the lowest effective dosage empirically, administration can be commenced at 20 ppm and then decreased gradually until efficacy (e.g., vasodilator efficacy) is lost. Where 20 ppm is deemed an insufficient inhaled dose, NO dosage may be increased gradually until effectiveness (e.g., vasodilator effectiveness) is observed. Such adjustment of dosage is routine for those of skill in the art. An advantage of the present invention is that in many cases it enables achievement of a desired therapeutic outcome at an NO dosage lower than that required if NO were administered alone. In addition, it may allow for the prolongation of an inhaled NO response, which may allow for intermittent therapy (i.e., increased intervals between inhalations of NO by a subject).

Inhaled NO can be administered from a source of stored, compressed NO gas. The source of NO can be 100% NO, or diluted with N₂ or any other inert gas (e.g., helium). The NO can be obtained and stored as a mixture free of any contaminating O₂ or higher oxides of nitrogen, because such higher oxides of nitrogen (which can form by reaction of O₂ with NO) are potentially harmful to lung tissues. If desired, purity of the NO may be demonstrated with chemiluminescence analysis, prior to administration to a patient. Chemiluminescence NO—NOₓ analyzers are commercially available (e.g., Model 14A, Thermo Environmental Instruments, Franklin, Mass.). The NO—N₂ mixture may be blended with air or O₂ through, for example, calibrated rotameters which have been validated previously with a spirometer. The final concentration of NO in the breathing mixture may be verified with a chemical or chemiluminescence technique (see, e.g., Fontijin et al., Anal. Chem. 42:575 (1970)). Alternatively, NO and NO₂ concentrations may be monitored by means of an electrochemical analyzer. Any impurities such as NO₂ can be scrubbed by exposure to NaOH solutions, baralyme, or sodalime. As an additional control, the FiO₂ of the final gas mixture may also be assessed. If desired, the ventilator may have a gas scavenger added to the expiratory outlet to ensure that significant amounts of NO will not escape into the adjacent environment.

In a hospital or emergency field situation, administration of NO gas could be accomplished, for example, by attaching a tank of compressed NO gas in N₂, and a second tank of oxygen or an oxygen/N₂ mixture, to an inhaler designed to mix gas from two sources; by controlling the flow of gas from each source, the concentration of NO inhaled by the patient can be maintained at an optimal level. NO gas may also be mixed with room air, using a standard low-flow blender (e.g., Bird Blender, Palm Springs, Calif.). NO may be generated from N₂ and O₂ (i.e., air) by using an electric NO generator. Such a generator is described in Zapol U.S. Pat. No. 5,396,882.

NO or CO may be provided intermittently from an inhaler. The use of an inhaler may be particularly advantageous if a compound that sensitizes soluble guanylate cyclase is administered, orally or by inhalation, in conjunction with the NO or CO.

Administration of a compound that sensitizes soluble guanylate cyclase may decrease the total dosage of NO or CO required (or allow intermittent dosage) to produce a satisfactory therapeutic or prophylactic effect. As a result of a decrease of NO or CO dosage or intermittent inhalations, an inhaler can be used less frequently. The compound that sensitizes soluble guanylate can be administered before (e.g., within 1, 12, or 24 hours before), during, or after (e.g., within 1, 12, or 24 hours after) inhalation of the gaseous NO or CO by the patient.

Inhaled NO or CO can optionally be administered by nasal prongs, mask, tent, intra-tracheal catheter or endotracheal tube, for an extended period, i.e., days or weeks. The administration may be continuous, during the extended period. Alternatively, administration could be intermittent during the extended period. The administration of gaseous NO or CO may be via spontaneous or mechanical ventilation.

Compounds that Sensitize Soluble Guanylate Cyclase

NO decomposes rapidly by reacting with molecular oxygen to produce nitrite and nitrate. In addition, NO entering the blood is rapidly inactivated by tight binding to hemoglobin. For these reasons, NO has only a short half-life in arterial blood. As detailed herein, compounds that sensitize soluble guanylate cyclase (sGC) to NO activation can augment and prolong the action of inhaled NO.

A compound that sensitizes soluble sGC can be introduced into a mammal by any suitable method, including via an oral, transmucosal, intravenous, intramuscular, subcutaneous, intraperitoneal, transcutaneous, or per rectum route. Alternatively, the compound can be inhaled by the mammal. For inhalation, the compound can be formulated as a dry powder or an aerosolized or nebulized solution having a particle or droplet size of less than 10 μm for optimal deposition in the alveoli, and may optionally be inhaled in a therapeutic gas containing NO.

Compounds that sensitize soluble guanylate cyclase (sGC) to NO activation can be identified by routine assays. Both in vitro cell free and cell based assays can be used to determine if a compound, such as an sGC activator, can sensitize sGC to activation by NO. In vitro kinetic assays can be performed using purified sGC, e.g., sGC purified according to Humbert, et al.,. Eur. J. Biochem. 190:273-278 (1990). An in vitro assay can be used to determine whether a compound potentiates NO stimulation of sGC, e.g., by determining whether the compound causes a leftward shift of the NO concentration-response curve of sGC activity (decreases EC₅₀) or increases the activity of sGC for any given dose of NO (Vmax). Examples of in vitro assays that can be used to show that a compound sensitizes sGC and causes a leftward shift of the concentration-response curve of sGC activity are described in Friebe et al., EMBO J. 15:6863-6868 (1996) and Friebe and Koesling, Mol. Pharmacol. 53:123-127 (1998).

Cell based assays employ cells with endogenous NO-sensitive sGC activity, such as platelets or aortic smooth muscle cells. In a platelet aggregation assay, the aggregation of stimulated platelets can be measured in the presence of an NO-donor alone, a candidate compound alone, or in the presence of both the candidate compound and the NO-donor. Platelet aggregation tracks cGMP production inside the cells, thus the synergistic effect of a compound and an NO-donor can be inferred from their combined effect on platelet aggregation. See, e.g., Friebe et al., Mol. Pharmacol. 54:962-967 (1998). Smooth muscle cell preparations can similarly be exposed to an NO-donor alone, to a compound alone, or to both the compound and the NO-donor. The accumulation of cGMP can then be measured to determine if a compound sensitizes sGC to an NO-donor. See, e.g., Mulsch et al., Brit. J. Pharmacol. 120:681-689 (1997).

Examples of compounds that sensitize sGC include 3-(5′-hydroxymethyl-2′-furyl)-1-benzylindazole (YC-1; Russwurm, J. Biol. Chem. 277:24883-24888, (2002), Schmidt et al., Mol. Pharmac. 59:220-224 (2001), Friebe, et al., Mol. Pharmacol. 54:962-967, (1998)) and compounds loosely based on YC-1 such as the pyrazolopyridine BAY 41-2272 (Stasch et al., Nature 410:212-215 (2001), the BAY 41-2272 derivatives ortho-(BAY 50-6038), meta-(BAY 51-9491) and para-PAL-(BAY 50-8364) (Becker et al., BMC Pharmacol. 1:13 (2001)), and BAY 41-8543 (Brit. J. Pharmacol. 135:333-343 (2002)). Additional compounds that stimulate sGC activation include 1-benzyl-3-(substituted hetaryl)-fused pyrazole derivatives (U.S. Pat. No. 6,180,656) and heterocyclylmethyl-substituted pyrazole derivatives (U.S. Pat. No. 6,166,027).

Routine sGC activity assays can be carried out to determine if an sGC activator also functions to sensitize sGC to NO. Exemplary activators of sGC include the substituted isoindolone derivatives described in U.S. Pat. No. 6,344,468, the sulfonylamino carboxylic acid N-arylamides described in U.S. Pat. No. 6,548,547, and the sulfur substituted sulfonylaminocarboxylic acid N-arylamides described in U.S. Pat. No. 6,335,334.

Assessment of Effects of Inhaled Nitric Oxide

When inhaled NO and a compound that sensitizes soluble guanylate cyclase are administered to treat or prevent a medical condition, it is in some cases desirable to monitor the effects of the administrations. Such monitoring can be used, in a particular individual, to verify desirable effects of the treatment. Such monitoring is also useful in adjusting the dose level, duration, and frequency of administration of inhaled NO in a given individual.

The effects of administration of inhaled NO and a compound that sensitizes soluble guanylate cyclase on a patient can be assessed by standard medical analyses used to evaluate the condition to be treated. For example, if the patient suffers from pulmonary vasoconstriction, pulmonary artery pressure can be monitored via a flow-directed pulmonary artery catheter, cardiac ultrasound, or range-gated doppler techniques. In another example, if the patient suffers from vascular thrombosis or arterial restenosis, these conditions can be monitored by examination of clinical manifestations such as chest pain, electrocardiography, serial analyses of vascular patency by ultrasound, or coronary angiography.

Other Agents Administered with Inhaled Nitric Oxide

In some embodiments, a phosphodiesterase inhibitor can be administered in conjunction with NO inhalation to inhibit the breakdown of cGMP by endogenous phosphodiesterases (see, e.g., U.S. Pat. Nos. 5,570,683 and 5,823,180). The phosphodiesterase inhibitor can be introduced into the mammal by any suitable method, including via an oral, transmucosal, intravenous, intramuscular, subcutaneous or intraperitoneal route. Alternatively, the inhibitor can be inhaled by the mammal. A suitable phosphodiesterase inhibitor is Zaprinast (M&B 22948; 2-o-propoxyphenyl-8-azapurine-6-one; Rhone-Poulenc Rorer, Dagenham Essex, UK).

An antithrombotic agent can be administered together with NO in certain methods described herein (e.g., treatment or prevention of ischemia-reperfusion injury or vascular thrombosis). Such antithrombotic agents serve to restore perfusion of the tissues susceptible to ischemia-reperfusion injury via thrombolysis, and augment the therapeutic effects of inhaled NO by decreasing the potential for activation of platelets in non-pulmonary tissues. Examples of antithrombotic agents are aspirin, streptokinase, urokinase, tissue plasminogen activator (“t-PA”), met-t-PA (i.e., t-PA with an N-terminal methionine residue), FEIX (a t-PA analog), heparin, hirudin, Hirulog (a hirudin analog), ticlopidine, and IIb/IIIa (e.g., Rheopro). One or more such antithrombotic agents may be administered to a mammal before, during, or after treatment with inhaled NO, so that the potential of platelets to become activated in non-pulmonary tissues is decreased.

The following are examples of the practice of the invention. They are not to be construed as limiting the scope of the invention in any way.

### EXAMPLES

### Example 1

### Pharmacological Sensitization of Soluble Guanylate Cvclase Produces Pulmonary Vasodilation and Modulates the Pulmonary Response to Inhaled Nitric Oxide

In awake lambs instrumented with vascular catheters and a tracheostomy tube, the thromboxane analog U-46619 was infused intravenously to increase mean pulmonary arterial pressure (PAP) to 35 mm Hg. After a stabilization period, seven animals received an intravenous infusion of BAY 41-2272 (Alexis Biochemicals, Lausen, Switzerland) at 0.03, 0.1, and 0.3 mg·kg⁻¹·h⁻¹ administered for 30 minutes each. In another eight animals, inhaled NO (2, 10, and 20 ppm) was administered in random order for 10 minutes each followed by 15 minute NO-free periods. An intravenous infusion of BAY 41-2272 (0.1 mg·kg⁻¹·h⁻¹) was then started. After increasing the infusion of U-46619 to maintain PAP at 35 mmHg, NO was inhaled as before BAY 41-2272. In an additional eight lambs, which received an intravenous infusion of the NO synthase inhibitor, L-NAME (25 mg/kg+5 mg·kg⁻¹·h⁻¹), together with U-46619, NO was inhaled before and during the infusion of BAY 41-2272.

Data were analyzed using repeated measures ANOVA followed by Dunnett adjustment. A p<0.05 was considered statistically significant.

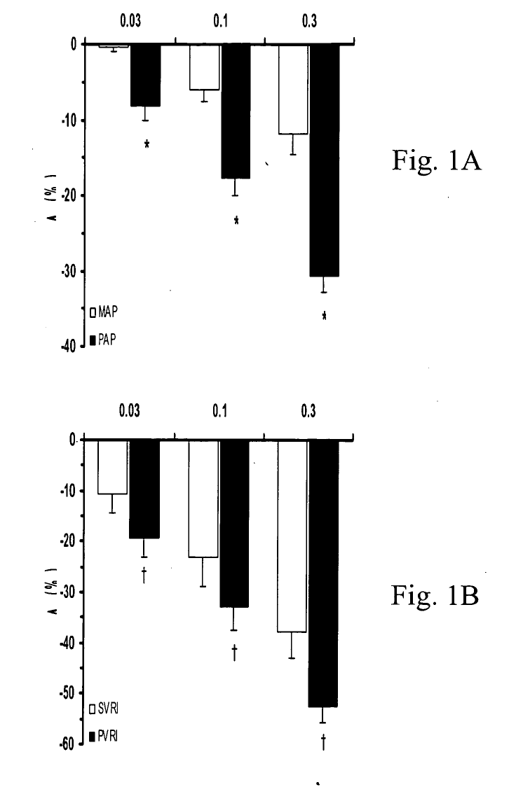

BAY 41-2272 produced dose-dependent reductions in mean PAP and pulmonary vascular resistance index (PVRI) that were significantly greater than the corresponding reductions in mean arterial pressure (MAP) and systemic vascular resistance index (SVRI) (FIGS. 1A-1B). Data are mean±SEM (n=7) and represent percent changes from the pre-treatment pulmonary hypertension values. * p<0.05 vs. MAP; † p<0.05 vs. SVRI.

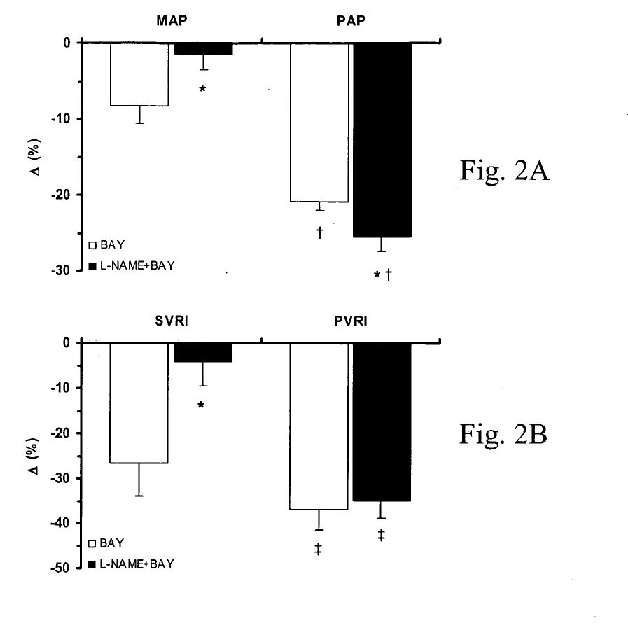

L-NAME abolished systemic but not pulmonary vasodilation induced by BAY 41-2272 (0.1 mg·kg⁻¹·h⁻¹) (FIGS. 2A-2B). Data are mean±SEM (n=8) and represent percent changes from the pre-treatment pulmonary hypertension values. * p<0.05 vs. BAY; † p<0.05 vs. MAP; ‡ p<0.05 vs. SVRI.

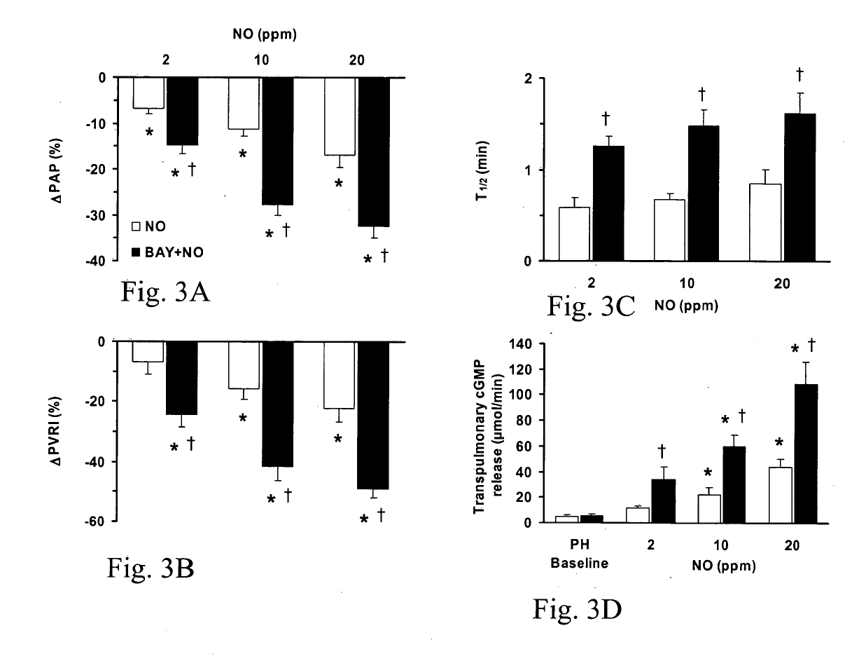

Inhaled NO produced a dose-dependent, selective pulmonary vasodilation (FIGS. 3A-3B). After NO was discontinued, PAP rapidly returned to baseline pulmonary hypertension (PH) with a T_{1/2}<1 min (FIG. 3C). Inhaled NO at 20 ppm also increased PaO₂/FiO₂ and reduced P(A-a)O₂ and Qs/Qt (P<0.05). Inhalation of 10 and 20 ppm of NO increased transpulmonary cGMP release (P<0.01) (FIG. 3D), whereas there were no significant changes of arterial methemoglobin concentrations.

Administration of BAY 41-2272 markedly enhanced the reductions of PAP, PVRI, and PVRI/SVRI induced by inhaled NO(P<0.05) (FIGS. 3A-3B). After NO was discontinued, the persistence of the pulmonary vasodilation (as reflected by T_{1/2}) during BAY 41-2272 infusion was greater than that before the infusion (P<0.05) (FIG. 3C). In the presence of BAY 41-2272, NO inhaled at 10 and 20 ppm produced minor reductions of pulmonary capillary wedge pressure (PCWP) and systemic vascular resistance index (SVRI) and increments in cardiac index (CI) and stroke volume index (SVI) (P<0.05 vs. baseline PH) and reduced right ventricle stroke work index (RVSWI) to a greater extent than did inhaled NO alone (P<0.05), whereas MAP, HR and central venous pressure (CVP) remained unchanged. Moreover, during BAY 41-2272 infusion, inhalation of 10 and 20 ppm NO augmented PaO₂/FiO₂ and reduced P(A-a)O₂ and Qs/Qt (P<0.01 vs. baseline PH). The co-administration of BAY 41-2272 and inhaled NO increased transpulmonary cGMP release to a greater extent than did inhaled NO alone (P<0.05) (FIG. 3D). During the period of NO administrations, the plasma BAY 41-2272 concentrations remained at a stable level.

### Example 2

### Sensitization of Soluble Guanylate Augments and Prolongs the Effects of Inhaled Nitric Oxide

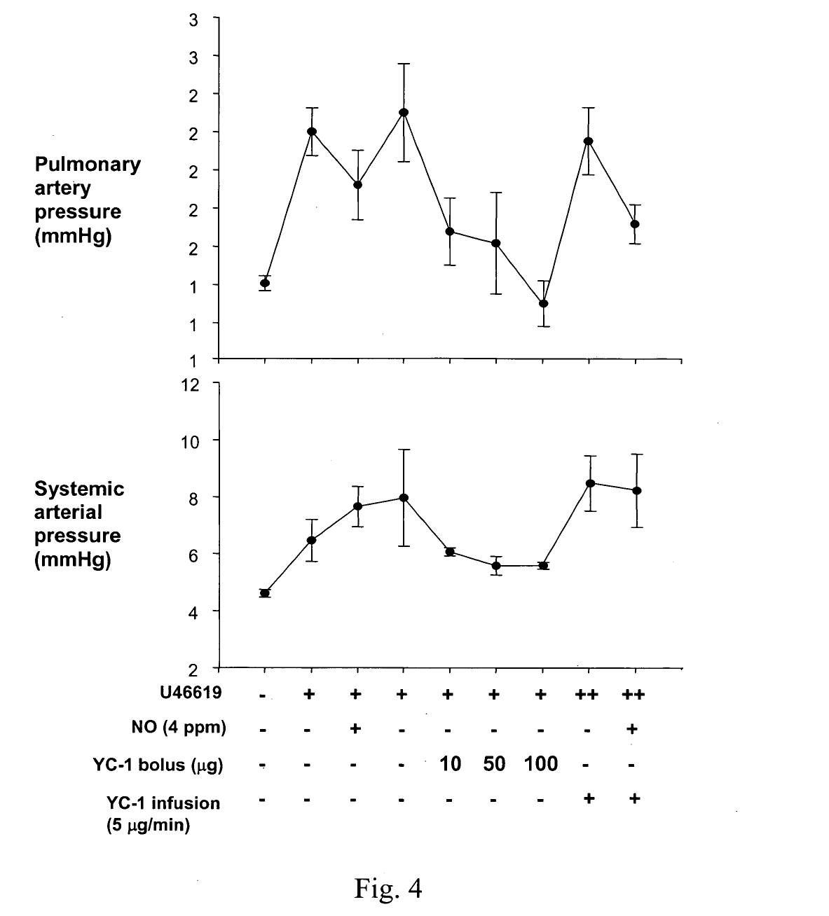

A mouse model of pulmonary hypertension was used to examine the combined effects of YC-1 and inhaled NO. Three mice (average weight 25 g) were anesthetized, their chest opened, catheters were placed in the carotid and pulmonary arteries, and pulmonary and systemic arterial pressures were recorded (FIG. 4). U46619, a thromboxane analogue was administered intravenously to induce pulmonary vasoconstriction. NO (4 parts per million) was added to the inspired gas, and pulmonary vasodilation was measured. Thereafter, NO inhalation was discontinued, and the duration of the pulmonary vasodilator effect was measured.

YC-1 was administered as boluses over one minute (10, 50 and 100 ug), and both pulmonary artery pressure and systemic blood pressure were measured. Thereafter, YC-1 was administered as an infusion (5 ug/min), and the rate of U46619 infusion was increased (2- to 3-fold) to re-establish pulmonary vasoconstriction. The pulmonary vasodilator response to breathing 4 ppm NO was measured, and duration of pulmonary vasodilation after discontinuing NO was assessed. Bolus administration of YC-1 induced both pulmonary and systemic vasodilation (FIG. 4).

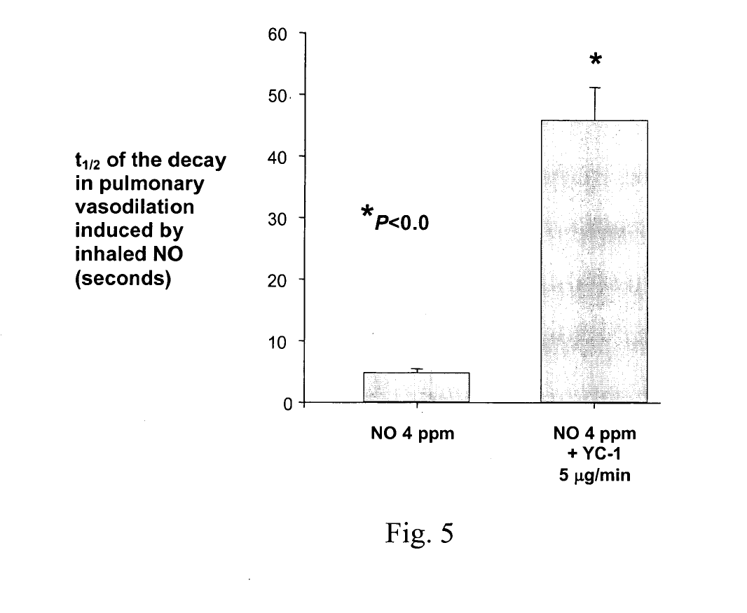

Breathing NO after YC-1 administration induced a greater pulmonary vasodilator response than did breathing NO alone. Addition of NO inhalation to YC-1 did not cause systemic vasodilation. Moreover, the duration of pulmonary vasodilation after discontinuing NO inhalation was greater during an infusion of YC-1 than before (P<0.01; FIG. 5).

### Example 3

### Effects of Carbon Monoxdie Inhaled Alone or in Combination with BAY 41-2272

Inhalation of CQ alone or following administration of BAY 41-2272 had no vasodilator effect on the U-46619-induced pulmonary vasoconstriction. Systemic hemodynamics, lung gas exchange, and transpulmonary cGMP release were also unchanged by CO inhalation. Arterial concentrations of carboxyhemoglobin gradually rose from 1.0±0.2% to 4.7±0.4% (P<0.01) and from 1.4±0.1% to 5.2±0.2% (P<0.01), respectively, after breathing 500 ppm CO alone or in combination with BAY 41-2272. The plasma levels of BAY 41-2272 remained stable during the period of CO administrations.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for enhancing the therapeutic or prophylactic effectiveness of inhaled nitric oxide, the method comprising:
identifying a mammal that has or is at risk of developing a condition amenable to treatment or prevention by inhalation of gaseous nitric oxide;
administering to the mammal by inhalation a therapeutically effective amount of gaseous nitric oxide; and
administering to the mammal a composition comprising a compound that sensitizes soluble guanylate cyclase, wherein the composition comprises an amount of the compound sufficient to enhance the therapeutic or prophylactic effectiveness of the inhaled gaseous nitric oxide,
wherein the method does not comprise the administration to the mammal of superoxide dismutase.

2. The method of claim 1, wherein, prior to administering the gaseous nitric oxide and the composition, the mammal is diagnosed as having or being at risk of developing pulmonary vasoconstriction.

3. The method of claim 1, wherein the mammal has or is at risk of developing pneumonia, traumatic injury, aspiration or inhalation injury, fat embolism in the lung, acidosis, inflammation of the lung, adult respiratory distress syndrome, acute mountain sickness, post cardiac surgery acute pulmonary hypertension, persistent pulmonary hypertension of the newborn, perinatal aspiration syndrome, hyaline membrane disease, acute pulmonary thromboembolism, acute pulmonary edema, heparin-protamine reactions, sepsis, hypoxia, asthma, status asthmaticus, or hypoxia of the newborn.

4. The method of claim 1, wherein the mammal has or is at risk of developing chronic pulmonary hypertension, bronchopulmonary dysplasia, chronic pulmonary thromboembolism, idiopathic pulmonary hypertension, or chronic hypoxia.

5. The method of claim 1, wherein, prior to administering the gaseous nitric oxide and the composition, the mammal is diagnosed as having or being at risk of developing bronchoconstriction.

6. A method for enhancing the therapeutic or prophylactic effectiveness of inhaled nitric oxide, the method comprising:
identifying a mammal that has or is at risk of developing a non-pulmonary condition amenable to treatment or prevention by inhalation of gaseous nitric oxide;
administering to the mammal by inhalation a therapeutically effective amount of gaseous nitric oxide; and
administering to the mammal a composition comprising a compound that sensitizes soluble guanylate cyclase, wherein the composition comprises an amount of the compound sufficient to enhance the therapeutic or prophylactic effectiveness of the inhaled gaseous nitric oxide.

7. The method of claim 6, wherein, prior to administering the gaseous nitric oxide and the composition, the mammal is diagnosed as having or being at risk of developing a vascular thrombosis.

8. The method of claim 6, wherein, prior to administering the gaseous nitric oxide and the composition, the mammal is diagnosed as having or being at risk of developing an acute ischemic coronary syndrome.

9. The method of claim 6, wherein, prior to administering the gaseous nitric oxide and the composition, the mammal is diagnosed as having or being at risk of developing arterial restenosis.

10. The method of claim 6, wherein, prior to administering the gaseous nitric oxide and the composition, the mammal is diagnosed as having or being at risk of developing a hemoglobinopathy.

11. The method of claim 6, wherein, prior to administering the gaseous nitric oxide and the composition, the mammal is diagnosed as having or being at risk of developing an ischemia-reperfusion injury.

12. The method of claim 6, wherein, prior to administering the gaseous nitric oxide and the composition, the mammal is diagnosed as having or being at risk of developing inflammation.

13. The method of claim 6, wherein the method does not comprise the administration to the mammal of superoxide dismutase.

14. A method of improving gas exchange in the lungs of a mammal, the method comprising:
identifying a mammal for whom an improvement in gas exchange within the lungs would be beneficial;
administering to the mammal by inhalation a therapeutically effective amount of gaseous nitric oxide; and
administering to the mammal a composition comprising a compound that sensitizes soluble guanylate cyclase, wherein the composition comprises an amount of the compound sufficient to enhance the therapeutic effectiveness of the inhaled gaseous nitric oxide and improve gas exchange in the lungs of a mammal,
wherein the method does not comprise the co-administration of superoxide dismutase.

15. The method of claim 14, wherein the mammal is hypoxic.

16. The method of claim 1, wherein the composition is inhaled in a gas comprising the gaseous nitric oxide.

17. A method for enhancing the therapeutic or prophylactic effectiveness of inhaled carbon monoxide, the method comprising:
identifying a mammal that has or is at risk of developing a condition amenable to treatment or prevention by inhalation of gaseous carbon monoxide;
administering to the mammal by inhalation a therapeutically effective amount of gaseous carbon monoxide; and
administering to the mammal a composition comprising a compound that sensitizes soluble guanylate cyclase, wherein the composition comprises an amount of the compound sufficient to enhance the therapeutic or prophylactic effectiveness of the inhaled gaseous carbon monoxide.

18. The method of claim 17, wherein, prior to administering the gaseous carbon monoxide and the composition, the mammal is diagnosed as having or being at risk of developing an ischemia-reperfusion injury.

19. The method of claim 17, wherein, prior to administering the gaseous carbon monoxide and the composition, the mammal is diagnosed as having or being at risk of developing inflammation.

20. The method of claim 17, wherein, prior to administering the gaseous carbon monoxide and the composition, the mammal is diagnosed as having a hemoglobinopathy.

21. The method of claim 17, wherein the method does not comprise the administration to the mammal of superoxide dismutase.

22. The method of claim 17, wherein the composition is inhaled in a gas comprising the gaseous carbon monoxide.

23. The method of claim 1, wherein the compound that sensitizes soluble guanylate cyclase is 3-(5′-hydroxymethyl-2′-furyl)-1-benzylindazole (YC-1).

24. The method of claim 1, wherein the compound that sensitizes soluble guanylate cyclase is 3-(4-amino-5-cyclopropylpyrimidine-2-yl)-1-(2-fluorobenzyl)-1H-pyrazolo[3,4-b]pyridine (BAY 41-2272).

25. The method of claim 1, wherein the mammal is a human.
